(19) United States
(12) Patent Application Publication
(10) Pub. No.: US 2010/0204582 A1
(43) Pub. Date: Aug. 12, 2010

(54) Multidimensional, multilayer ultrasound transducer probe for medical ultrasound imaging

(76) Inventor: Xuan-Ming Lu, Issaquah, WA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/369,913

(22) Filed: Feb. 12, 2009

(51) Int. Cl. A61B 8/14

(52) U.S. Cl. 600/459

(57) ABSTRACT

By using larger segments for transmit than receive in ultrasound imaging, the number of transmit beamformer channels relative to receive beamformer channels is reduced. The space and power requirements of the transmit beamformer channels are reduced, assisting in placement within a transducer probe. The larger segments for transmit are obtained by interconnecting electrodes used for transmit on different elements. Each element includes two or more layers of transducer material and a corresponding three or more electrodes. One of the electrodes is a transmit electrode. The transmit electrodes of two or more elements are connected together, such as sharing a via connection to a transmit beamformer channel. Receive electrodes for each element are isolated from each other and connect to receive beamformer channels. The multi-layer structure of the elements provides for transmit grouping of elements and separate reception without grouping or with different grouping.

### BACKGROUND

The present invention relates to transducer arrays, such as multi-dimensional transducer arrays. In particular, multi-dimensional transducer arrays for medical diagnostic imaging are provided.

Ultrasound transducers connect through cables with imaging systems. For linear arrays, 64, 128, or 256 elements connect through separate cables to the imaging system. A similar number of transmit and receive beamformer channels are provided for generating acoustic transmit beams and receiving samples of a scanned region. To avoid electrical cross-talk and other interference, coaxial cables are used for communicating from the transducer array to the imaging system. As the number of elements increases, the number of coaxial cables increases. However, miniaturization of coaxial cables is expensive and limited.

For multi-dimensional arrays, such as two-dimensional arrays, the number of elements may be drastically increased as compared to one-dimensional arrays. A corresponding increase in the number of transmit and receive beamformers channels is expensive and results in bulky or unusable cables. One approach to limit the number of cables is to use one set of elements, such as a grouping of elements sparsely distributed on the array, for transmit and a different set of elements for receive operations. A fewer number of transmit elements are provided, resulting in a fewer number of transmit beamformer channels. However, providing separate transmit and receive elements may adversely affect the received signals and require extra elements.

In the ultrasound system, a transmit/receive (T/R) switch separates each channel into receive and transmit channels. The T/R switch is a passive switch, such as disclosed in U.S. Pat. No. 6,269,052. Active switching elements, such as a high-voltage CMOS analog switch, may not settle very rapidly. This makes it difficult to switch them during a scan line, such as between transmit and receive events for scanning a line. Operating active switching elements also requires carefully timed control signals from the ultrasound system.

Bi-layer transducers provide a variety of operating modes not available in conventional transducers. By driving the two layers of an element in phase and then summing the received signals of the two layers out of phase, a harmonic mode is provided. The transmitted acoustic field has low second harmonic content, and received energy includes information at the second harmonic with fundamental frequency suppression.

### BRIEF SUMMARY

By way of introduction, the preferred embodiments described below include methods, systems, transducer arrays, and probes for medical ultrasound imaging. By using larger segments for transmit than receive, the number of transmit beamformer channels relative to receive beamformer channels is reduced. Where the transmit waveform generators of the transmit beamformer channels are positioned within an ultrasound probe, the space and power requirements of the transmit beamformer channels are reduced based on the reduction in number of transmit segments.

The larger segments for transmit are obtained by interconnecting electrodes used for transmit on different elements. Each element includes two or more layers of transducer material and a corresponding three or more electrodes. One of the electrodes is a transmit electrode. The transmit electrodes of two or more elements are connected together, such as sharing a via connection to a transmit beamformer channel. Receive electrodes for each element are isolated from each other and connect to receive beamformer channels. The multi-layer structure of the elements provides for transmit grouping of elements and separate reception without grouping or with different grouping.

In a first aspect, a multi-dimensional transducer system for medical ultrasound imaging is provided. A plurality of elements are spaced in a multi-dimensional grid. Each of the elements includes at least first and second layers of transducer material and at least first, second, and third electrically separate electrodes. A first of the electrodes is between the first and second layers of transducer material. An electrical connection is formed between the first electrodes of at least first and second elements of the plurality of elements. A transmit beamformer channel electrically connects with the first electrodes and electrical connection of the first and second elements such that the first and second elements together generate an acoustic waveform. First and second receive beamformer channels connect with the first and second elements such that signals generated by both the first and second elements are separately received.

In a second aspect, a multi-dimensional transducer array is provided for medical ultrasound imaging. A plurality of multiple transducer material layer elements are provided. The elements are grouped by commonly connected transmit electrodes. The elements have electrically separate receive electrodes. A via is provided for each of the groups of elements. The via intersects each of the elements in the respective group such that the transmit electrodes are commonly connected.

In a third aspect, a method is provided for medical ultrasound imaging. A transmit waveform is generated with a plurality of elements having transmit electrodes electrically connected together and having a plurality of transducer layers. Echo signals are received with electrically isolated receive electrodes of each the plurality of elements. The receive electrodes are separate from the transmit electrodes. The plurality of elements are grounded during the receiving and generating with a ground electrode separate from the transmit and receive electrodes.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed on illustrating the principles of the invention. Moreover, in the figures, like reference numerals designate corresponding parts throughout the different views.

FIG. 1 is a block diagram of one embodiment of a multi-dimensional transducer array system;

FIG. 2 is a perspective view of four multi-layer elements and electrodes in one embodiment;

FIG. 3 is a representation of embodiments of a via for interconnecting transmit electrodes in multi-layer elements;

FIG. 4 is a flow chart diagram of one embodiment of a method for medical ultrasound imaging with multi-layer elements;

FIG. 5 is graph of simulated transmit efficiency according to one embodiment;

FIG. 6 is graph of simulated receive signal-to-noise ratio according to one embodiment; and

FIG. 7 is a graph of measured bilayer frequency response according to one embodiment.

### DETAILED DESCRIPTION OF THE PRESENTLY PREFERRED EMBODIMENTS

Multi-layer (e.g., bi-layer) PZT elements are used in a multidimensional (e.g., 2D) transducer to increase harmonic imaging (e.g., tissue harmonic) sensitivity and penetration. The transmitter and receiver connect to separate electrodes, so no transmit/receive switch is needed. A via or other interconnection connects transmit electrodes from different elements together. For example, a single via intersects two or four elements, connecting the transmit electrodes of the elements in groups of two or four elements, respectively, for transmit operation. The transmit voltage may be reduced to half of a single layer for the same power. Denser logical chips (thinner silicon layer) may be provided. The number of vias and transmitters are reduced to four (or two) times more than for a fully sampled array for cost reduction and smaller transmit chip size. Two-way signal-to-noise (SNR) improvement may be about 5-10 dB.

The transducer array is used for abdominal and gastro-intestinal volumetric imaging or 3D/4D cardiology. Due to reduction in size, application specific integrated circuits or other transmit or receive beamformer components may fit inside a transesphogeal probe.

The transducer system may be used for other applications, such as any 2D, 3D, or 4D ultrasound applications. One alternative application for the bi-layer transducer is multi-frequency mode imaging. The low frequency (e.g. f0=2.5 MHz) fundamental mode may be used for penetration, when applied electric fields are in the same direction as poling for the both layers. The low frequency fundamental mode is performed with the bottom electrode grounded and the center electrode used for transmission and reception at the fundamental frequency, f0. The high frequency (e.g. 2f0=5 MHz) fundamental mode may be used for high resolution. The high frequency fundamental mode is performed by floating the middle electrode, and the bottom electrode is used for transmission and reception at the high frequency.

Another alternative application for the bi-layer is HIFU or therapeutic ultrasound. The low frequency (f0) mode is for pushing or heating, and the high frequency (2f0) mode is for imaging.

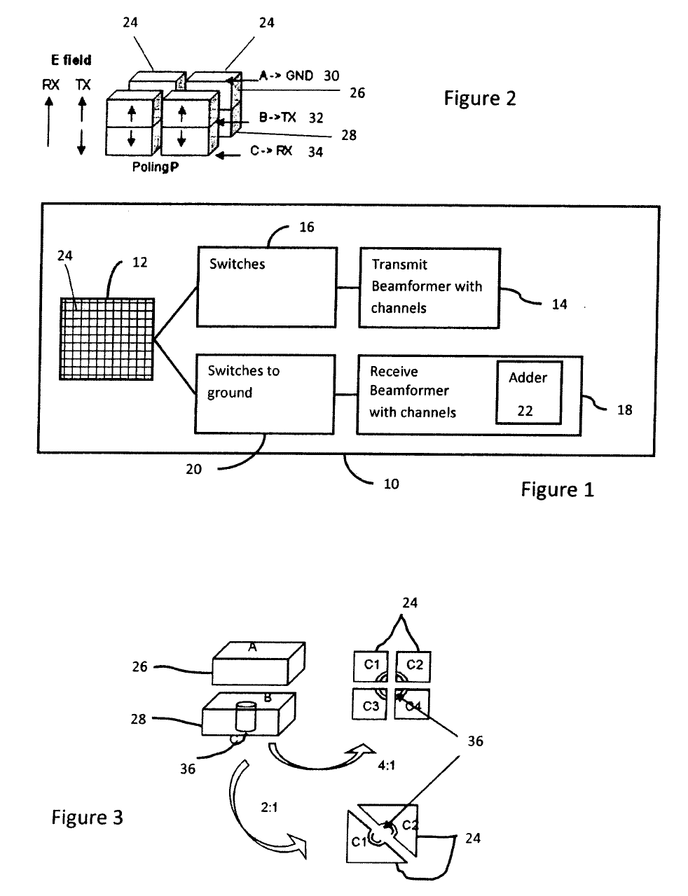

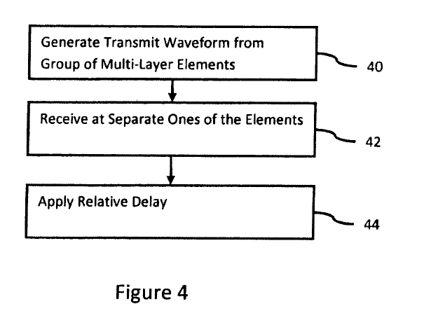

FIG. 1 shows a multi-dimensional transducer system for medical ultrasound imaging in one embodiment. The system includes an array 12 of elements 24, a transmit beamformer 14 with transmit beamforming channels 14, switches 16, a receive beamformer 18 with receive beamformer channels and an adder 22, and switches 20. As shown, the array 12 of elements, the transmit beamformer 14 and the receive beamformer 18 are positioned within the probe housing 10. In alternative embodiments, the transmit beamformer 14 and/or receive beamformer 18, or portions thereof, are positioned in an imaging system and connect with the array 12 of elements through a cable. The channels of the transmit beamformer 14 and receive beamformer 18 connect to the array 12 of elements 24 on separate paths or circuits. In alternative embodiments, the channels of the transmit beamformer 14 and receive beamformer 18 connect to the array 12 using a same path through a transmit/receive switch. The switches 16 and/or 20 are not provided in other embodiments.

The probe housing 10 is plastic, rubber, metal or now-known or later-developed material for at least partially housing the array 12. In one embodiment, the probe housing 10 is adapted for handheld use, such as being shaped and/or sized to have a grip or other portion for holding by the user. Alternatively, the housing 10 is shaped for use internally in a patient, such as an endoscope or catheter device. The probe housing 10 includes an acoustic window, such as a polymer, plastic, glass or air window for allowing transmission of acoustic energy from the array 12 into a patient. In one embodiment, the probe housing 10 or the system use the components, structure, or materials disclosed in U.S. Pat. No. 6,994,674, the disclosure of which is incorporated herein by reference. For example, the transmit beamformer 14 and receive beamformer 18 of the system of FIG. 1 use the transmit and receive beamformer structures disclosed in the above-referenced patent. The position of the transmit beamformer channels and receive beamformer channels relative to the probe housing 10 are also the same as in the above-referenced patent. In alternative embodiments, the position of the components, the components, or other aspects are different.

The array 12 is a multi-dimensional transducer array for medical ultrasound imaging. The array 12 includes a plurality of elements 24 positioned in a multi-dimensional grid pattern. As shown in FIG. 1, the multi-dimensional grid pattern in one embodiment has elements 24 spaced along a plurality of columns and rows. In the embodiment, each of the elements 24 is spaced along the grid pattern with half-wavelength spacing. A desired imaging frequency is used to determine the distance from the center of each element 24 to adjacent elements. Greater or lesser wavelength spacings may be provided. Each of the elements 24 are separated by a kerf or other electrical and/or physical separation. In one embodiment, the multi-dimensional grid pattern is a two-dimensional grid pattern, but any rectangular, square, hexagonal or other shaped grids may be used. For example, the array 12 is a 1.25, 1.5, 1.75 or 2-dimensional array. As another example, the array 12 has one or more rows or columns with fewer elements 24 than other rows or columns.

Each of the elements 24 is a piezoelectric or capacitive membrane-based transducer of acoustic and electrical energies. Alternatively, other now-known or later-developed materials or structures for transducing between acoustic and electrical energy may be used. In one embodiment, each of the elements 24 is a composite material of piezoelectric and a polymer, silicon, rubber or other bonding material for holding posts or fragments of the piezoelectric in positions relative to each other. The kerfs separating each of the elements 24 are filled with air, rubber, silicon, polymer or other now-know or later-developed material.

FIG. 2 shows four elements 24 of the array 12. The array 12 may have hundreds or thousands of elements 24. Each of the transducer elements 12 has a top layer 26 and a bottom layer 28 of piezoelectric or other transducer material. The layers 26, 28 may both be between a backing layer and a matching layer. Additional layers 26, 28 may be provided, such as three or more layers of transducer material. The layers 26, 28 are stacked along a range dimension or direction of propagation. Each layer 26, 28 has the same or different elevation and azimuth extent and shape.

The layers 26 and 28 are a same or different piezoelectric material, such as a piezoelectric single crystal, piezoelectric ceramic or piezoelectric polymer material, or their composites with epoxy or other filler materials. In alternative embodiments, the one or more of the layers 26, 28 are electrostatic micromachined devices, such as electrostatic moving membrane devices. In yet other embodiments, the one or more of the layers 26, 28 are electrostrictive material, such as PMN-PT. Each of the layers 26, 28 has a same or different geometry and/or material. For example, the same thickness is used for each layer, such as a ½ mm or 315 μm thickness. Other thicknesses may be used, including thicknesses that vary in one or more dimensions.

In one embodiment, the top and bottom layers 26, 28 have different transducer materials. For example, the bottom layer 28 is a solid piezoelectric material, such as a solid ceramic or electrostatic substrate. The solid piezoelectric material is free of epoxy or free from kerfs for each transducer element. The top layer 26 is piezo-composite material, such as a combination of piezoelectric ceramic and epoxy or polymer. Piezo-composite materials include piezoelectric material beams separated by epoxy-filled kerfs in one dimension or piezoelectric material posts separated by epoxy-filled kerfs in two dimensions, but other piezo-composites may be used. In one example embodiment, the top layer 26 is a piezo-composite having 14-19 Mrayl acoustic impedance, and the bottom layer 28 is a solid piezoelectric material having about 30 Mrayl acoustic impedance.

If the transducer material is piezoelectric, the transducer material of the layers 26, 28 is poled. The poling is along or substantially parallel to the propagation direction. In one embodiment, the different layers have substantially opposite poling directions. For example, FIG. 2 shows the opposite poling represented by the arrows in the elements 24. In other embodiments, two or more layers 26, 28 are poled in a same direction.

The electrodes 30, 32, 34 are metal, but other conductors may be used. Sheets with or without flexible circuit material (e.g., polyester film) form the electrodes 30, 32, 34. Alternatively, the electrodes 30, 32, 34 are deposited material. The electrodes 30, 32, 34 are formed as part of the stack, such as with sintering, or are separate layers, such as with asperity contact. In one embodiment, the center electrode 32 is formed in the stack by sintering or asperity contact, but the outer electrodes 30, 34 are formed in the stack with asperity contact and bonding.

Each layer 26, 28, is associated with two electrodes 30, 32, 34. The top layer 26 has electrodes 30, 32 on opposite sides. The bottom layer 28 has electrodes 32, 34 on opposite sides. The center electrode 32 is a single electrode shared by both layers 26, 28. Alternatively, the center electrode 32 is formed from two electrodes in contact with each other.

The electrodes 30, 32, and 34 are electrically separate from each other. The transducer material separates the electrodes 30, 32, 34, allowing three different independent connections to each element 24. The electrodes 30, 32, 34 connect with wires, traces, or other conductors for routing signals to or from the electrodes 30, 32, 34.

In one embodiment, one or more of the electrodes 30, 32, 34 have a fixed or non-switched connection. For example, the top or outside electrode 30 of the top layer 26 has a fixed connection to a local ground. The top electrodes 30 are shorted to ground all of the time. The top electrode 30 may be a sheet of conductive material covering a plurality of elements 24, such as associated with a ground plane. In alternative embodiments, a switched connection to ground is used. The top electrode 30 is positioned closest to a patient during use. Alternatively, one, more, or all of the connections of the electrodes 30, 32, 34 are switched, such as with passive and/or active switching. Switched connections to ground may be used.

Other electrodes 30, 32, and/or 34 are switched or fixed. Switches may be used for aperture control or selection. In another example, the electrode 32 between the layers 26, 28 is switchably connected by the switches 16 from an open or floating connection to a connection with a channel of the transmit beamformer 14. During transmit operation, the switch 16 connects the middle electrode 32 to the transmit beamformer 14. During receive operation, the switch 16 causes the middle electrode 32 to float. The middle electrode 32 is not connectable with the ground electrode 30 or the receiving electrode 34. The system is free of a transmit/receive switch, passive switching, or other connection between the middle electrode 32 and other electrodes 30, 34. In alternative embodiments, the middle electrode 32 is unswitched, such as being connected to the transmit beamformer channel.

As another example, the bottom electrodes 34 of the elements 24 connect with respective receive beamformer channels of the receive beamformer 18. A separate receive beamformer channel is provided for each element 24, allowing relative delays and/or apodization across the elements 24. In the example of FIG. 2, four receive beamformer channels connect to the four elements 24. The bottom electrodes 34 may be switched between the receive beamformer channels during reception and ground during transmission. In alternative embodiments, no switching is provided.

To reduce the number of transmit beamformer channels for a given array, the electrodes 32 used for transmit operation may be combined across elements 24. An electrical connection 36 between the middle electrodes 32 or a transmit electrode of at least two elements electrically connects the elements 24 to act as a larger transmit element. A single transmit beamformer channel drives the two or more electrically interconnected elements 24. Any conductive connection may be used, with or without switching. The electrical connection 36 is direct, such as without any passive or active switching. In other embodiments, indirect connection is provided through one or more switches.

For the array 12, the elements 24 are used individually or singly during receive. One or more elements 24 may be connected by switching to form larger receive elements 24, but the separate elements 24 are capable of use individually by the bottom electrodes 34 being electrically separate from each other. In other embodiments, two or more elements 24 have fixedly connected bottom or other receive electrodes 34.

For transmit on the array 12, the elements 24 are grouped by commonly connected transmit electrodes 32. For example, groups of two or more elements 24 have electrically interconnected middle electrodes 32. FIG. 3 shows a group of four elements 24 with an interconnection 36 of the transmit electrodes 32. FIG. 3 also shows a group of two elements 24 with interconnection 36 of the transmit electrodes 32. The same size and grouping arrangement is repeated across the array 12. Alternatively, different groupings (different in size and/or arrangement, such as 2×2 or 1×4) are provided in a same array 12.

The ratio of transmit channels to receive channels is different. For example, four receive channels are provided for every transmit channel in the example of FIG. 2. Since the transmit electrodes 32 are connected by the electrical connection 36, only one transmit beamformer channel connects to the elements 24 of that grouping. In the other example shown in FIG. 3, the ratio is 2 to 1. Other ratios may be provided, such as equal (i.e., 1 to 1) or more transmit channels. Using fewer transmit channels due to the electrical connection 36 within the array 12 and within the elements 24 may reduce the size of high voltage integrated circuits to implement the transmit beamformer channels. The electrical connection 36 provides a multilayer two-dimensional matrix for tissue harmonic or other types of imaging with a transmit to receive channel ratio of 4:1, 2:1 or other ratio.

The electrical connection 36 is fixed or part of the structure of the elements 24. For example, the middle electrodes 32 are deposited or layered and not separated by kerfs between the elements 24 of a group. As another example, wire jumpers connect the middle electrodes 32 within a group. In another example, one or more vias route the middle electrodes 32 of a group to a common signal trace, such as on a flexible circuit. Separate vias are provided for the separate groups of elements 24.

FIG. 3 shows one embodiment of a via as the electrical connection 36. The via is formed by laser drilling, chemical etching, or other micromachining technology and then filling with conductive epoxy. The via is large enough to intersect the different elements in the group. Larger vias, such as 50-100 μm, may be more easily formed. The via connects the middle electrodes 32 to the bottom of the elements 24 for electrical contact with the transmit beamformer channel. The via commonly connects the transmit electrodes 32 of the elements 24 in the group. For a four element 24 grouping, the via may intersect the adjacent corners of the elements 24. For a two element 24 grouping, the via intersects any adjacent surfaces of the elements 23. The via is formed at the center of the group of elements 24, but may be formed at other locations. More than one via may be provided.

The via is formed in the bottom layer 28 and not the top layer 26. The layers 26, 28 may be separately formed and processed before stacking for bonding and/or sintering. In alternative embodiments, the via is formed in multiple layers 26, 28. Dicing, patterned deposition, or other techniques may be used to isolate the via from the ground electrodes 30 and the receiving electrodes 34.

The via and the receive electrodes 34 connect to the switches 16 and 20 and/or beamformers 14, 18 with conductors, coaxial cables, traces, signal paths, or combinations thereof. In one embodiment, the via and receive electrodes 34 connect with single sided or double sided flexible circuit material with traces connected to the switches 16 and 20.

The switches 16 and 20 connect the electrodes 32, 34 with the appropriate or separate transmit and receive beamformer channels. The switches 16, 20 comprise transistors, multiplexers, or other switches. A switch 16, 20 is provided for each beamformer channel. The switches 16, 20 may additionally select apertures or connect different elements 24 to different beamformer channels at different times.

The switches 16 connect the electrical connection 36 to a transmit beamformer channel or provide an open disconnect (float the transmit electrodes 32). During reception, the switches 16 disconnect the electrical connection 36. During transmission, the switches 16 connect the electrical connection to the transmit beamformer 14.

The switches 20 connect the receive electrodes 34 of the elements 24 to a respective number of receive beamformer channels or ground the receive electrodes 34. During reception, the switches 20 connect the receive electrodes 34 to the receive beamformer 18. During transmission, the switches 20 connect the receive electrodes 34 to ground or other fixed potential.

A transmit beamformer channel of the transmit beamformer 14 electrically connects with the transmit electrodes 32 through the electrical connection 36 of the grouped elements 24. This common connection allows the elements 24 of the group to generate an acoustic waveform together. Other groups of elements 24 are connected with other transmit beamformer channels for generating other relatively delayed and apodized acoustic waveforms. The different acoustic waveforms constructively interfere to form a transmit beam along one or more scan lines. Since the elements 24 are grouped for transmission, the number of transmitters is less (e.g., 4× or 2× less) than receive beamformer channels (e.g., 2048/4=>512). Using fewer transmit beamformer channels may reduce the transmit chip area. Since the transmit function uses higher voltage (e.g., tens or hundreds of volts), this reduction may provide a greater power and/or area reduction than reducing low voltage reception channels.

The transmit beamformer channel is an analog or digital transmit beamformer channel. For example, a transmit beamformer disclosed in U.S. Pat. Nos. 5,675,554, 5,608,690, 6,005,827, or 6,104,670, the disclosures of which are herein incorporated by reference, is used. Other sources of waveforms may be used, such as waveform generators, pursers, switches, a waveform memory, mixer, or digital-to-analog converter. The waveform for a given transmit beamformer channel is delayed and amplified relative to other transmit beamformer channels.

The transmit beamformer channels are electrical traces connecting between a transmit waveform generator and the array 12. In other embodiments, the transmit beamformer channels include delays, timing circuits, amplifiers, waveform generators, or combinations thereof for generating relatively delayed and apodized waveforms for each of a plurality of transmit element groups on the array 12. In one embodiment, the transmit beamformer channels are implemented on one or more application-specific integrated circuits, processors, field-programmable gate arrays, digital circuits, analog circuits, combinations thereof, or other now-known or later-developed devices within the probe housing 10. Alternatively, a portion or all of the transmit beamformer channels other than the traces or signal lines are positioned outside of the transducer probe housing 10. In one embodiment, the waveform generators are transistors, networks or other devices for generating unipolar or bipolar waveforms.

The transmit beamformer channels connect with respective transmit groups of elements 24 through the corresponding electrical connection 36. All of the groups together form a transmit aperture. Different relatively delayed or apodized waveforms are applied to different transmit groups for generating a beam, fan or other distribution of acoustic energy with the array 12.

The receive beamformer channels connect with the elements 24 such that signals generated by the elements 24 are separately received. The receive beamformer channels are analog or digital receive beamformer channels. For example, a receive beamformer 18 disclosed in U.S. Pat. No. 5,685,308, the disclosure of which is incorporated herein by reference, is used. The receive beamformer channel includes a delay, phase rotator, summer, and/or filter for relatively delaying and apodizing signals from different channels and then summing the signals with the adder 22.

The receive beamformer channels are signal traces, amplifiers, delays, summers, multipliers, phase rotators, digital circuits, analog circuits, combinations thereof, or other now-known or later-developed receive beamformer channels. In one embodiment, each receive beamformer channel within the probe housing 10 includes signal traces to different receive electrodes 34 with or without a multiplexer or other switching, preamplifiers, and a multiplexer for applying time-division multiplexing to a plurality of receive beamformer channels. Alternatively, one or more summers for partial or complete beamforming are within the probe housing 10. The signals from the plurality of channels are multiplexed onto a same signal line for later demultiplexing and beamforming. Additional, or fewer components of the receive beamformer channels may be included within the transducer probe housing 10. As used herein, a receive beamformer channel may include only receive signal lines for outputting data to the receive beamformer 18. Likewise, a transmit beamformer channel may include only signal lines for connection with the transmit beamformer 14. Each of the receive beamformer channels or a subset of the channels are connected or connectable with different receive electrodes 34.

A fewer number of transmit beamformers channels are used for the transmit aperture than receive beamformer channels used for receive aperture. To minimize the spatial requirements of the array, the transmit and receive apertures share at least some or all of the elements 24 in common. By reducing the number of transmit groups and associated transmit beamformer channels, fewer components and space are used for transmit beamformer channels within the probe housing 10. In one embodiment, the number of transmit beamformer channels are fewer by a multiple of 2, 4, other integer number or a non-integer number than the number of receive beamformer channels.

In one example, the elements 24 have half-wavelength spacing based on the receive frequency. For harmonic imaging, the transmit or fundamental frequency may be less, such as ½ of the receive harmonic frequency. The combination of elements 24 for transmit may correspond to half-wavelength spacing for the lower frequency transmit operation. The transmit groups have a one-wavelength spacing in the grid of the array 12. The receive elements 24 have a one-half wavelength spacing within the grid. Other spacings may be provided. Other multiples than four may be used.

An optional filter may be included in the receive beamformer 18 or separate from the receive beamformer 18. The filter provides highpass, bandpass, lowpass, or spectral whitening response. The filter passes information associated with the desired frequency band, such as the fundamental transmit frequency band, a harmonic of the fundamental frequency band, or any other desired frequency band. As used herein, harmonic comprises higher harmonics (e.g., second, third, . . . ), fractional harmonics (3/2, 5/3, . . . ), or subharmonics (½, ⅓, . . . ). The filter may comprise different filters for different desired frequency bands or a programmable filter. For example, the filter demodulates the signals to base band. The demodulation frequency is programmably selected in response to the fundamental center frequency or another frequency, such as a second harmonic center frequency. Signals associated with frequencies other than near the base band are removed by low pass filtering. As another example, the filter provides band pass filtering.

As an additional or alternative option, a memory, phase rotator, amplifier (e.g., multiplier) and/or summer are provided. By combining received signals responsive to different transmit events with relative phasing and/or weighting, information at desired frequencies may be isolated or enhanced relative to other frequencies.

The adder 22 is a summer, cascade of summers, or other circuit for adding signals or data from different receive beamformer channels. The adder 22 is within the probe housing 10, but may be located in an imaging system remote or outside of the probe housing 10.

In the embodiments discussed above, the transmit and receive apertures have a same area, such as using all of the same elements 24. In alternative embodiments, the same area is provided for each of the transmit and receive apertures, but with some different elements 24. For example, the transmit aperture may be shifted or include different sparse sampling than the receive aperture. At least some overlap of the transmit and receive apertures is provided.

In an alternative or additional embodiment, the number of transmit beamformer channels is reduced through a different transmit aperture size. Using groupings of elements for transmit and single elements or smaller groupings on receive, a lesser area for the transmit aperture uses fewer transmit beamformer channels than a larger area receive aperture uses receive beamformer channels.

Using a same or similar size transmit aperture as receive aperture free of sparse sampling may provide advantages. Where each of the transmit and receive apertures are a same size or use the entire array 12, better signal to noise and lateral resolution may be provided. For tissue or contrast agent harmonic imaging, the acoustic energy is transmitted at about half of the center frequency of the transducer array 12 (e.g., 2 MHz). The half of the center frequency used on transmit corresponds to a half-wavelength sampling of transmit groups of elements using four elements 24 each. During receive operation, a half-wavelength sampling between receive elements is provided using individual ones of the elements 24. The second harmonic (e.g., 4 MHz) of the transmitted fundamental frequency corresponds to half-wave length sampling of the elements 24. As a result, the clutter level and grating lobe interference may be reduced. Other harmonics, including fractional or sub-harmonics may be used. For sub-harmonics, the transmit groups may be smaller than the receive groups. For fundamental imaging, an imaging frequency is selected in between the half-wavelength spacing of the transmit groups and the half-wavelength spacing of the elements. Alternatively, the imaging frequency is selected independent of the wavelength spacing or based on the wavelength spacing of either the transmit groups or the receive elements 24. Since a grating lobe may be at different angles given the different spacing, the resulting two-way grating lobe interference may be minimal. Other advantages, different advantages, only one of the advantages discussed above or none of the advantages discussed above may be provided.

FIG. 3 shows one embodiment of a method for medical ultrasound imaging. The method may reduce transmit and/or receive channels in a multi-dimensional transducer array. The method of FIG. 3 is implemented using the structure or components of FIGS. 1-3, but different structures or components may be used in other embodiments. In one embodiment, the number of transmit beamformer channels and associated transmit groups of elements is less than the number of receive beamformer channels and associated receive elements. An electrical connection within the array and/or within the elements groups the elements for transmit operation. For receive operation, the elements transduce independently. Different array external or internal connections grouping different elements than for transmit may be used for receive operation.

The top electrode or ground plane remains grounded during both transmit and receive operations. The top electrode is isolated from the separate transmit and receive electrodes. The ground connection may be fixed or switched. For example, diodes or other devices ground the electrodes. In one embodiment, a flexible circuit is coated or formed with a ground plane. The single ground plane is then switched to ground without requiring a switch for each element. Alternatively, the ground plane is connected to a local or other ground without switching. For transmit operation, the receive electrodes are grounded using switches, passive circuits (e.g., diodes), or other devices. The transmit electrodes are not connectable to ground in one embodiment, but may be grounded in other embodiments.

A transmit aperture is formed. The transmit aperture has a plurality, N, of transmit groups of elements on a multi-dimensional transducer array. In one embodiment, one or more of the transmit groups includes a plurality of elements, such as groups of two or four elements. Groups of elements are connected together. The connection is provided as part of the physical structure in multi-layer elements. For example, the electrodes between transducer layers are connected with other elements in each group. A transmit beamformer channel is configured to connect to at least two or more elements, the elements in a group. Different elements are used for different groups and different transmit beamformer channels.

In act 40, transmit waveforms are generated. Each of the groups of elements receives an electrical waveform from a respective transmit beamformer channel or other transmitter. A separate waveform is applied for each group of elements of the transmit aperture. The waveforms may be different, such as associated with different apodization weighting or relative delays, but may be the same. In response to application of the waveforms to different groups of elements, a transmit beam or fan of acoustic energy is generated by the transducer array. In one embodiment, the transmit waveforms are generated in a probe. For example, transmit waveform generators are provided in a handheld transducer probe with the array. Alternatively, the transmit waveforms are generated in an imaging system and provided through one or more cables to the array.

Each electrical waveform is applied to the group through a common electrical connection. The elements are separated by kerfs and/or electrode patterns. For transmission, the groups of elements include two or more elements having a common connection, such as a via extended to a middle or internal electrode of multilayer elements. The via may be large enough to intersect and electrically connect different elements in the group. The electrical waveform passes through the via common to each of the elements of the group to the transmit electrodes of the elements. Using multilayer elements allows for common connection on transmit and separate operation on receive. Since the elements of each group are electrically connected together, the group of elements acts as a single transmit element.

During transmission, the receive electrodes of the multilayer elements are grounded. Alternatively, the receive electrodes are disconnected or float. Other electrodes may be grounded or float as well. For a two layer element embodiment, the middle electrodes receive the transmit waveforms. The electrodes on the top and bottom or opposite sides of the elements are grounded. For example, the top electrode is part of a grounding plane connected to ground for both transmit and receive operation. In alternative embodiments, the middle electrodes are receive electrodes and the bottom electrodes are transmit electrodes. The via connects the receive electrodes.

In the bi-layer embodiment shown in FIG. 2, two layers of PZT transducer material are used for transmitting at a fundamental frequency or band and for second harmonic reception. During transmit, the electric field generated by applying the electrical waveform to the middle electrode is in the same direction or parallel with the poling. Both layers are synchronized as one thick layer.

After transmission, a receive aperture is formed. The receive aperture has a plurality, M, of receive elements or element groupings on the multi-dimensional transducer array. The number, M, of receive elements is greater than the number, N, of transmit groups, but may be less or the same. For receive, the elements may operate independently. Alternatively, a different electrical connection within the array is used than for the transmit groups. Each receive beamformer channel connects to a respective receive group. For example, receive beamformer channels connect to individual elements. A same element may be used for both transmit and receive operation while a different element is used for a separate reception but a same transmit group.

In act 42, echo signals are received. Acoustic echoes are received by the array using the receive electrodes. The receive electrodes are electrically isolated from each other for every element or differently than the transmit electrodes. In the embodiment of FIG. 2, each element operates independently, so each receive electrodes generates a separate electrical waveform. To allow different groupings of elements for transmit and receive operation, the electrodes of the multilayer elements are separate. The transmit electrodes are isolated from the reception electrodes. Electrical signals generated by the elements in response to the acoustic echoes are transmitted from the electrodes to the receive beamformer channels.

In the example of FIG. 2, each element transduces the echo signals into electrical signals separately. The element acts as one thin layer to receive at the second harmonic of the fundamental transmit frequency. Other harmonics or the fundamental may be used for reception. The transmit or middle electrodes are disconnected or float. The top or outside electrode is grounded. The potential between the electrodes on opposite sides (e.g., top and bottom electrodes) operates the element as an element with a single layer or fewer number of layers than for transmit or actually exist. The electric field extends across both layers. On the bottom layer, the electric field and poling are opposite. In alternative embodiments, the electric field and poling are opposite in the top layer or are the same for both layers.

For transmission, other groups of elements also generate acoustic waves. The acoustic waves may be relatively apodized and/or delayed. The elements of the groups also receive echo signals. The groups and elements of the transmit and receive apertures, respectively, are used by the transmit and receive beamformers to form transmit and receive beams.

In act 44, part of the receive beamforming is provided by applying relative delays to the electrical signals of the elements. Since each element separately transduces the echoes, the resulting electrically signals may be separately delayed by time delay and/or phase shift. In other embodiments, the different relative delays are applied to groups of elements. For example, the different elements may be connected to together to form a receive element group. As another example, different elements may operate with the same delay due to the steering angle, so may be switchably connected and use a same beamformer channel. In one partial beamforming embodiment, the delays and summing are applied in a sub-array process in a probe, and different delays and summing are applied, in an imaging system, to sub-array sums.

The array may be manufactured using any possible technique. In one embodiment represented in FIG. 3, vias are formed in a slab of piezoelectric material. The vias are filled with conductive epoxy. Another slab without vias is provided. The slabs are coated on both sides with conductive electrode material, such as using deposition techniques. One slab may be coated on just one side. A bottom flexible circuit for the receive and transmit apertures, backing, the slabs of transducer material and matching layers are stacked and laminated or bonded. Dicing cuts then form kerfs along the azimuth and elevation dimensions, forming the elements. The kerfs intersect the vias. A top flexible circuit is then applied to form the ground plane. Separate patterned traces in the bottom flexible circuit connect with the remaining via material and the receive or bottom electrodes.

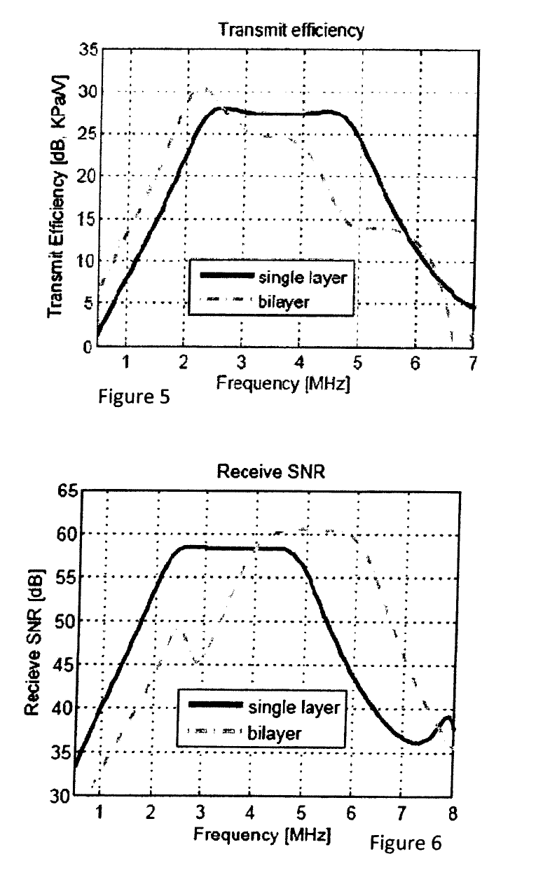

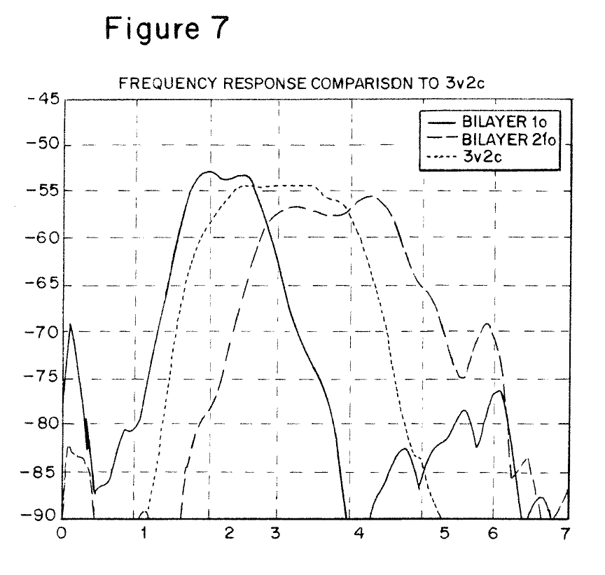

FIG. 5 shows a simulation of the transmit efficiency, and FIG. 6 shows a simulation of receive SNR for a 2D array (0.2×0.2 mm pitch, 4 MHz) single layer (400 μm) vs. bilayer (2×315 μm). The transmit efficiency increased by 5 dB at 2.5 MHz. 3^{rd} harmonic operation may be available (RX 6 MHz). The receive SNR increased more than 5 dB for 5 MHz and more than 10 dB for 6 MHz. FIG. 7 shows a similar trend in frequency response comparison between the measured biayer harmonic responses and the operation of a 3V2c (1D single layer) transducer.

While the invention has been described above by reference to various embodiments, it should be understood that many changes and modifications can be made without departing from the scope of the invention. It is therefore intended that the foregoing detailed description be regarded as illustrative rather than limiting, and that it is the following claims, including all equivalents, that are intended to define the spirit and the scope of this invention.

I (we) claim:

1. A multi-dimensional transducer system for medical ultrasound imaging, the multi-dimensional transducer system comprising:
a plurality of elements spaced in a multi-dimensional grid, each of the elements including at least first and second layers of transducer material and at least first, second, and third electrically separate electrodes, a first of the electrodes being between the first and second layers of transducer material;
an electrical connection between the first electrodes of at least first and second elements of the plurality of elements;
a transmit beamformer channel electrically connected with the first electrodes and electrical connection of the first and second elements such that the first and second elements together generate an acoustic waveform; and
first and second receive beamformer channels connected with the first and second elements such that signals generated by both the first and second elements are separately received.

2. The multi-dimensional transducer system of claim 1 wherein the electrical connection comprises a via.

3. The multi-dimensional transducer system of claim 2 wherein a single via is formed in the first and second elements, the via formed in the first layers and not the second layers of the first and second elements.

4. The multi-dimensional transducer system of claim 2 wherein the at least first and second elements comprise the first, the second, a third, and a fourth element, the via formed at a corner of the first and not the second layer of each of the first, second, third, and fourth elements.

5. The multi-dimensional transducer system of claim 1 wherein further transmit beamformer channels connected with further groups of at least two of the plurality of elements and further receive beamformer channels connect separately to each of the plurality of elements.

6. The multi-dimensional transducer system of claim 1 wherein the second electrodes of the plurality of elements are operable to connect with ground, and wherein the third electrodes of the plurality of elements connect with respective receive beamformer channels, the third electrodes of the first and second elements connected with the first and second receive beamformer channels.

7. The multi-dimensional transducer system of claim 1 further comprising:
a first switch operable to connect and disconnect the transmit beamformer channel from the first electrodes of the first and second elements, the first switch operable to disconnect during reception by the first and second receive beamformer channels and connect during transmission; and
second and third switches operable to connect the second electrodes of the first and second elements to ground during transmission and to the first and second receive beamformer channels, respectively, during reception;
wherein the first electrodes are not able to be connected with the second and third electrodes, the system free of a transmit and receive switch.

8. The multi-dimensional transducer system of claim 1 wherein the first and second layers are oppositely poled.

9. The multi-dimensional transducer system of claim 1 further comprising an adder of a receive beamformer, the adder operable to add signals from the first and second receive beamformer channels, the adder being within a transducer probe housing.

10. A multi-dimensional transducer array for medical ultrasound imaging, the multi-dimensional transducer array comprising:
a plurality of multiple transducer material layer elements, the elements grouped by commonly connected transmit electrodes, the elements having electrically separate receive electrodes; and
a via for each of the groups of elements, the via intersecting each of the elements in the respective group such that the transmit electrodes are commonly connected.

11. The multi-dimensional transducer array of claim 10 wherein the via is through one of the multiple transducer material layers of the elements and not through another one of the multiple transducer material layers.

12. The multi-dimensional transducer array of claim 10 further comprising a ground plane connected with the plurality of elements.

13. The multi-dimensional transducer array of claim 10 wherein the multiple transducer material layers comprise oppositely poled piezoelectric layers.

14. A method for medical ultrasound imaging, the method comprising:
generating a transmit waveform with a plurality of elements having transmit electrodes electrically connected together and having a plurality of transducer layers;
receiving echo signals with electrically isolated receive electrodes of each the plurality of elements, the receive electrodes separate from the transmit electrodes; and
grounding the plurality of elements during the receiving and generating with a ground electrode separate from the transmit and receive electrodes.

15. The method of claim 14 wherein generating comprises applying an electrical waveform through a common via to the transmit electrodes of the plurality of elements, the transmit electrodes being between the transducer layers.

16. The method of claim 14 wherein receiving comprises transducing the echo signals into electrical signals separately by each of the elements, the electrical signals on the receive electrodes below a bottom one of the transducer layers; and
further comprising applying different relative delays to the electrical signals of each element.

17. The method of claim 14 further comprising repeating the generating and receiving for other groups of elements, the generating for the plurality of elements and other groups of elements forming a transmit beam, and the receiving for the plurality of elements and other groups of elements being for a receive beam.

18. The method of claim 14 wherein generating comprises grounding the receive electrodes and the ground electrode on opposite sides of the elements and applying an electrical waveform to the transmit electrodes between the transducer layers.

19. The method of claim 14 wherein receiving comprises disconnecting the transmit electrodes between the transducer layers, grounding the ground electrode, and receiving with the receive electrode, the ground electrode and the receive electrode on opposite sides of the elements.

20. The method of claim 14 wherein generating comprises passing an electrical waveform through a via common to each of the elements to the transmit electrodes.
